(19) United States
(12) Patent Application Publication
(10) Pub. No.: US 2005/0152861 A1
(43) Pub. Date: Jul. 14, 2005

(54) Cosmetic antiperspirant roll-on formulations based on a w/o emulsion

(76) Inventors: Stefan Bruening, Duesseldorf (DE); Achim Ansmann, Erkrath (DE); Helga Gondek, Duesseldorf (DE)

Correspondence Address: COGNIS CORPORATION;PATENT DEPARTMENT, 300 BROOKSIDE AVENUE, AMBLER, PA, 19002, US

(21) Appl. No.: 10/495,697

(22) PCT Filed: Nov. 8, 2003
(86) PCT No.: PCT/EP02/12485

(30) Foreign Application Priority Data
Nov. 17, 2001 (DE) 101 56 666.2

(51) Int. Cl. A61K 7/36

(52) U.S. Cl. 424/67

(57) ABSTRACT

An antiperspirant composition containing: (a) an antiperspirant component; (b) a polyolpoly-12-hydroxystearate; (c) an oil component; and (d) water, and wherein the composition is a water-in-oil emulsion having a viscosity of from about 500 to 10,000 mPas at 20° C.

### FIELD OF THE INVENTION

This invention relates to antiperspirant roll-on formulations based on water-in-oil emulsions.

### PRIOR ART

Antiperspirants are available on the market in many forms, for example as sprays, creams, sticks and roll-on formulations based on o/w emulsions.

Antiperspirant roll-on formulations based on o/w emulsions are attended by the disadvantage that they often leave white residues, i.e. tend to form microfoams, and have stability problems.

W/O antiperspirant formulations based on certain alums are known from EP 998 909 A1. Unfortunately, these known formulations are limited in their deodorant performance. A perspiration-inhibiting water-resistant cream is known from DE 24 08 663. U.S. Pat. No. 5,534,246 describes water-in-oil antiperspirant roll-ons which contain polyoxyethylated C₆₋₂₂ fatty alcohols, ethoxylated alkylphenols and polyethylene glycol ethers of methyl glucose or sorbitol as emulsifiers. These formulations are in need of improvement in regard to their long-term stability, particularly at elevated temperatures.

Accordingly, the problem addressed by the present invention on the one hand was to reduce white residues from antiperspirant roll-on formulations, i.e. to provide low-viscosity formulations which would have a reduced tendency to form microfoam and, despite the presence of antiperspirant components, would show high dermatological compatibility. In addition, the formulations would have reduced tackiness and would feel dry on the skin, would be rapidly absorbed and, despite high salt levels, would show very high stability, even under temperature stress.

### DESCRIPTION OF THE INVENTION

The present invention relates to w/o emulsions with a viscosity of 500 to 10,000 mPa·s at 20° C. (Brookfield RVT, spindle TA, 20 r.p.m., with Helipath) containing (a) at least one antiperspirant component, (b) at least one w/o emulsifier selected from the group of polyolpoly-12-hydroxystearates, (c) an oil phase and (d) water.

It has surprisingly been found that antiperspirant roll-on formulations based on w/o emulsions show distinctly reduced microfoam formation by comparison with o/w emulsions. Through the use of special w/o emulsifiers, namely polyolpoly-12-hydroxystearates, the compositions according to the invention have excellent stability despite comparatively high concentrations of the antiperspirant components, i.e. despite high salt levels, and show high dermatological compatibility. The w/o emulsions according to the invention are used in roll-on applicators. In a preferred embodiment, they have a viscosity of 500 to 5,000 mPa·s at 20° C. (Brookfield RVT, spindle TA, 20 r.p.m., with Helipath).

Antiperspirant Components

According to the invention, water-soluble, astringent metallic salts, more particularly inorganic and organic salts of aluminium, zirconium and zinc or mixtures of these salts, for example aluminium chlorohydrates and/or aluminium zirconium chlorohydrates, are suitable as antiperspirant components. These antiperspirants probably act by partially blocking the sweat glands through the precipitation of proteins and/or polysaccharides. Besides the chlorohydrates, aluminium hydroxylactates and acidic aluminium/zirconium salts may also be used. For example, an aluminium chlorohydrate which corresponds to the formula [Al₂(OH)₅Cl].2.5H₂O and which is particularly preferred for the purposes of the invention is commercially available under the name of Locron® from Clariant GmbH. The aluminium zirconium tetrachlorohydrex glycine complexes marketed, for example, by Reheis under the name of Rezal® 36G are also preferably used in accordance with the invention. The preparations according to the invention preferably contain at least one aluminium salt and, more particularly, an aluminium zirconium tetrachlorohydrex glycine complex as their antiperspirant component. The aluminium salt is present in concentrations of normally 1 to 30% by weight, preferably 5 to 25% by weight and more particularly 5 to 20% by weight.

Polyolpoly-12-hydroxysearates

The polyolpoly-12-hydroxystearates which form component (b) are known substances, cf. in particular European patent EP 0 766 661 B1. The polyol component of these emulsifiers may be derived from substances containing at least 2, preferably 3 to 12 and more particularly 3 to 8 hydroxyl groups and 2 to 12 carbon atoms. Typical examples are:
(a) glycerol and polyglycerol;
(b) alkylene glycols such as, for example, ethylene glycol, diethylene glycol, propylene glycol;
(c) methylol compounds such as, in particular, trimethylol ethane, trimethylol propane, trimethylol butane, pentaerythritol and dipentaerythritol;
(d) alkyl glucosides containing 1 to 22, preferably 1 to 8 and more particularly 1 to 4 carbon atoms in the alkyl group, for example methyl and butyl glucoside;
(e) sugar alcohols containing 5 to 12 carbon atoms, for example sorbitol or mannitol,
(f) sugars containing 5 to 12 carbon atoms, for example glucose or sucrose;
(g) aminosugars, for example glucamine.

Among the emulsifiers suitable for use in accordance with the invention, reaction products based on polyglycerol and polyethylene glycol are particularly important by virtue of their excellent applicational properties. It has proved to be of particular advantage to use reaction products of poly-12-hydroxystearic acid with polyglycerols which have the following homolog distribution (the preferred ranges are shown in brackets):

| | | |
|---|---|---|
| glycerols | 5 to 35 (15 to 30)% | by weight |
| diglycerols | 15 to 40 (20 to 32)% | by weight |
| triglycerols | 10 to 35 (15 to 25)% | by weight |
| tetraglycerols | 5 to 20 (8 to 15)% | by weight |
| pentaglycerols | 2 to 10 (3 to 8)% | by weight |
| oligoglycerols | to 100% | by weight |

According to the invention, it is of particular advantage to use the poly(12-hydroxystearic acid)polyglycerol ester which is marketed under the name of Dehymuls® PGPH (INCI: Polyglyceryl-2-Dipolyhydroxystearate) by Cognis Deutschland GmbH & Co. KG. A PEG-30 dipolyhydroxystearate may also be used with advantage. In a particularly preferred embodiment, the composition according to the invention contains a combination of a PEG-30 Dipolyhydroxystearate and a poly(12-hydroxystearic acid)polyglycerol ester, i.e. for example Arlacel® P 135 and Dehymuls® PGPH. Accordingly, the present invention also relates to the use of polyolpoly-12-hydroxystearates for reducing microfoam formation in antiperspirant formulations.

The polyolpoly-12-hydroxystearates are present in the emulsions according to the invention in a quantity of normally 1 to 10% by weight, preferably 2 to 8% by weight and more particularly 3 to 7% by weight.

Oil Components

The w/o emulsions according to the invention contain an oil phase which may be made up of a single oil component or of a mixture of oil components. In the context of the invention, oil components are substances which are liquid at 20° C. and immiscible with water at 25° C. or mixtures of such substances. The oil components are present in the emulsions according to the invention in quantities of typically 5 to 30% by weight, preferably 5 to 25% by weight and more particularly 10 to 20% by weight.

Suitable oil components are, for example, Guerbet alcohols based on fatty alcohols containing 6 to 18 and preferably 8 to 10 carbon atoms (for example Eutanol® G), esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₃₋₃₈ alkylhydroxycarboxylic acids with linear or branched C₆₋₂₂ fatty alcohols, more especially diethyl hexyl malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates such as, for example, Dicaprylyl Carbonate (Cetiol® CC), Guerbet carbonates based on fatty alcohols containing 6 to 18 and preferably 8 to 10 carbon atoms, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group such as, for example, Dicaprylyl Ether (Cetiol® OE), ring opening products of epoxidized fatty acid esters with polyols (Hydagen® HSP, Sovermol® 750, Sovermol® 1102), silicone oils (cyclomethicone, silicon methicone types, etc.) and/or aliphatic or naphthenic hydrocarbons such as, for example, mineral oil, Vaseline, petrolatum, squalane, squalene or dialkyl cyclohexanes.

A distinct improvement in the sensory properties of the w/o emulsions according to the invention, more particularly faster absorption of the antiperspirant salt compared with o/w roll-on emulsions, reduced tackiness and a lower skin irritation potential, is achieved when dialkylethers and/or dialkyl carbonates are used as oil components either on their own or in combination with other oil components. In a preferred embodiment of the invention, the oil phase contains at least one oil component selected from the group of dialkyl(ene) ethers, dialkyl(ene) carbonates or a mixtures of these substances.

The dialk(en)yl ethers may be symmetrical or nonsymmetrical, branched or unbranched, saturated or unsaturated. Saturated C₆₋₃₂ dialkylethers such as, for example, di-n-octyl ether, di-(2-ethylhexyl)-ether, lauryl methyl ether or octyl butyl ether and didoceyl ether are particularly suitable for the purposes of the invention. The compounds may be prepared from fatty alcohols in the presence of acidic catalysts using generally known processes, cf. for example DE 19511668 A1, DE 19831705 A1 and DE 19943585. Typical examples of such ethers are products obtained by etherification of caproic alcohol, caprylic alcohol, 2-ethylhexyl alcohol, capric alcohol, lauryl alcohol, myristyl alcohol, cetyl alcohol, palmitoleyl alcohol, stearyl alcohol, isostearyl alcohol, elaidyl alcohol, petroselinyl alcohol, linolyl alcohol, linolenyl alcohol, oleyl alcohol, ricinolyl alcohol, elaeostearyl alcohol, arachidyl alcohol, gadoleyl alcohol, behenyl alcohol, erucyl alcohol and brassidyl alcohol, Guerbet alcohols and technical mixtures thereof which are obtained, for example, in the high-pressure hydrogenation of technical methyl esters based on fats and oils.

The dialk(en)yl carbonates may be symmetrical or non-symmetrical, branched or unbranched, saturated or unsaturated. Among the dialk(en)yl carbonates, linear or branched, saturated or unsaturated C₆₋₃₂ dialkyl(ene) carbonates, for example dihexyl, dioctyl, di-(2-ethylhexyl)- or dioleyl carbonate, are preferred for the purposes of the invention. The compounds may be obtained by transesterification of dimethyl or diethyl carbonate with corresponding hydroxy compounds using known methods as reviewed in Chem. Rev. 96, 951 (1996). Typical examples of alkyl(ene) carbonates are transesterification products of dimethyl and/or diethyl carbonate with caproic alcohol, caprylic alcohol, 2-ethylhexyl alcohol, capric alcohol, lauryl alcohol, myristyl alcohol, cetyl alcohol, palmitoleyl alcohol, stearyl alcohol, isostearyl alcohol, elaidyl alcohol, petroselinyl alcohol, linolyl alcohol, linolenyl alcohol, oleyl alcohol, ricinolyl alcohol, elaeostearyl alcohol, arachidyl alcohol, gadoleyl alcohol, behenyl alcohol, erucyl alcohol and brassidyl alcohol, Guerbet alcohols and technical mixtures thereof which are obtained, for example, in the high-pressure hydrogenation of technical methyl esters based on fats and oils.

The addition of silicone compounds to the compositions according to the invention creates a relatively light sensory impression on application. Accordingly, another preferred embodiment of the invention is characterized in that the oil phase contains at least one silicone compound which is liquid at 20° C.

Suitable silicone compounds are, for example, dimethyl polysiloxanes and cyclic silicones and optionally substituted analogs thereof. These include, for example, DM-Fluid® 0.6cs, DM-Fluid® 1.0cs, DM-Fluid® 1.5 cs, DM-Fluid® 2.0cs, DM-Fluid® 10cs, DM-Fluid® 100cs, DM-Fluid® 500cs, which are marketed by Shin Etsu Silicones of America, Inc.

For example, cyclomethicones and dimethicones may be used in quantities of 0.5 to 20% by weight, preferably in quantities of 1 to 15% by weight and more particularly in quantities of 1 to 13% by weight, based on the composition as a whole. The addition of dimethicones contributes towards further reducing the formation of microfoam.

A preferred embodiment of the w/o emulsion contains (a) 1 to 30% by weight of at least one aluminium salt, (b) 1 to 10% by weight of at least one w/o emulsifier selected from the group of esters of 12-hydroxystearic acid with polyethylene glycols or polyglycerols or a mixture of these esters, (c) 5 to 30% by weight of an oil phase and (d) 40 to 90% by weight of water. The w/o emulsion preferably contains (a) 5 to 25% by weight of at least one aluminium salt, (b) 2 to 8% by weight of at least one w/o emulsifier selected from the group of esters of 12-hydroxystearic acid with polyethylene glycols or polyglycerols or a mixture of these esters, (c) 5 to 25% by weight of an oil phase and (d) 40 to 80% by weight of water. A particularly preferred w/o emulsion contains (a) 5 to 20% by weight of at least one aluminium salt, (b) 3 to 7% by weight of at least one w/o emulsifier selected from the group of esters of 12-hydroxystearic acid with polyethylene glycols or polyglycerols or a mixture of these esters, (c) 10 to 20% by weight of an oil phase and (d) 40 to 70% by weight of water. The oil phase preferably contains dialkyl carbonates, dialkylethers or a mixture of these substances, in a particularly preferred embodiment in combination with a silicone compound which is liquid under normal pressure at 20° C.

Additional Deodorant Components

The w/o emulsions according to the invention may contain additional deodorant components, such as esterase inhibitors for example. The esterase inhibitors are preferably trialkyl citrates, such as trimethyl citrate, tripropyl citrate, triisopropyl citrate, tributyl citrate and, in particular, triethyl citrate (Hydagen® CAT, Cognis Deutschland GmbH). Esterase inhibitors inhibit enzyme activity and thus reduce odor formation. The free acid is probably released through the cleavage of the citric acid ester, reducing the pH value of the skin to such an extent that the enzymes are inhibited. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester. Antibacterial agents which influence the germ flora and destroy or inhibit the growth of perspiration-decomposing bacteria, may also be present in the compositions. Examples of such antibacterial agents are chitosan, phenoxyethanol and chlorhexidine gluconate. 5-Chloro-2-(2,4-dichlorophenoxy)-phenol, which is marketed under the name of Irgasan® by Ciba-Geigy of Basel, Switzerland, has also proved to be particularly effective.

Bactericidal or bacteriostatic components may additionally be present to improve the deodorant effect. These include, for example, chitosan, phenoxyethanol and 5-chloro-2-(2,4-dichlorophenoxy)-phenol, which is marketed by Ciba-Geigy of Basle, Switzerland, under the name of Irgasan®, and antibacterial perfumes such as, for example, thymol, thyme oil, eugenol, clove oil, menthol, mint oil, farnesol, and antibacterial glycerol esters such as, for example, glycerol monocaprate, glycerol monocaprylate, glycerol monolaurate (GML) and diglycerol monocaprate (DMC).

The addition of odor absorbers can also increase the deodorant performance. Odor absorbers are not active against bacteria, but merely reduce the partial pressure of the odor-forming components. Their principal component is, for example, a complex zinc salt of ricinoleic acid or special perfumes of substantially neutral odor which are known to the expert as “fixateurs”.

Suitable perspiration-absorbing substances are, for example, modified starch such as, for example, Dry Flo® Plus (National Starch), silicates and talcum. Suitable odor-masking perfumes are vegetable, animal and synthetic perfumes.

Humectants/Skin Moisturizers

Accordingly, in another preferred embodiment, the w/o emulsion contains at least one humectant. Humectants contribute towards improving the low-temperature stability of the w/o emulsions according to the invention. Humectants are present in quantities of normally 0.1 to 10% by weight, preferably 0.5 to 10% by weight and more particularly 0.5 to 6% by weight, based on the w/o emulsion.

Suitable humectants are inter alia amino acids, pyrrolidone carboxylic acid, lactic acid and salts thereof, lactitol, urea and urea derivatives, uric acid, glucosamine, creatinine, cleavage products of collagen, chitosan or chitosan salts/derivatives and, in particular, polyols and polyol derivatives (for example glycerol, diglycerol, triglycerol, ethylene glycol, propylene glycol, butylene glycol, erythritol, 1,2,6-hexanetriol, polyethylene glycols, such as PEG-4, PEG-6, PEG-7, PEG-8, PEG-9, PEG-10, PEG-12, PEG-14, PEG-16, PEG-18, PEG-20), sugars and sugar derivatives (inter alia fructose, glucose, maltose, maltitol, mannitol, inositol, sorbitol, sorbityl silanediol, sucrose, trehalose, xylose, xylitol, glucuronic acid and salts thereof), ethoxylated sorbitol (Sorbeth-6, Sorbeth-20, Sorbeth-30, Sorbeth-40), honey and hydrogenated honey, hydrogenated starch hydrolyzates and mixtures of hydrogenated wheat protein and PEG-20-acetate copolymer. According to the invention, particularly preferred humectants are glycerol, diglycerol and triglycerol.

Silicas

A further improvement in the sensory properties (particularly dry skin feel) can be achieved by incorporation of silicas by which are meant compounds with the general formula SiO₂.nH₂O. Highly disperse silicas, more particularly Aerosil®, are preferred for the purposes of the invention. Accordingly, in another preferred embodiment, the w/o emulsion contains silicas, preferably in quantities of 0.1 to 3.0% by weight.

Alkali Metal/Alkaline Earth Metal Salts

Another preferred embodiment of the w/o emulsion according to the invention contains at least one inorganic salt selected from alkali metal or alkaline earth metal chlorides or sulfates or a mixture of these salts. This contributes towards increasing the stability of the emulsion and the low-temperature stability of the w/o emulsion according to the invention. This is presumably attributable to the fact that migration of the emulsifier from the boundary layer into the inner aqueous phase is distinctly reduced by the salt content. Water-soluble salts such as, for example, sodium chloride, sodium sulfate, potassium sulfate, magnesium chloride, calcium chloride or magnesium sulfate or mixtures of these salts may be used in accordance with the invention.

Other Auxiliaries and Additives

The w/o emulsions according to the invention may contain a number of other auxiliaries and additives such as, for example, other emulsifiers, pearlizing waxes, consistency factors, thickeners, superfatting agents, stabilizers, polymers, fats, waxes, lecithins, phospholipids, biogenic agents, antioxidants, antidandruff agents, film formers, swelling agents, insect repellents, self-tanning agents, tyrosine inhibitors (depigmenting agents), hydrotropes, solubilizers, preservatives, perfume oils, dyes, etc.

### EXAMPLES 1-7 ACCORDING TO THE INVENTION AND COMPARISON EXAMPLE C1

| | INCI | C1 | 1 | 2 | 3 | 4 | 5 | 6 | 7 |
|---|---|---|---|---|---|---|---|---|---|
| Composition | | | | | | | | | |
| (% by weight) | | | | | | | | | |
| Eumulgin S2 | Steareth-2 | 3.0 | | | | | | | |
| Eumulgin S21 | Steareth-21 | 2.0 | | | | | | | |
| Dehymuls ® | Polyglyceryl-2 | | 2.0 | 2.0 | | 2.0 | 2.0 | 2.0 | 2.0 |
| PGPH | Dipolyhydroxystearate | | | | | | | | |
| Arlacel ® P 135 | PEG-30 | | 2.0 | 2.2 | 4.4 | 2.2 | 2.2 | 2.2 | 2.2 |
| | Dipolyhydroxystearate | | | | | | | | |
| Cetiol ® OE | Dicaprylyl Ether | 1.0 | 4.5 | 4.5 | 4.5 | 4.5 | 4.5 | 4.5 | 4.5 |
| Cetiol ® CC | Dicaprylyl Carbonate | 1.0 | 4.5 | 4.1 | 3.7 | 4.1 | 4.1 | 4.1 | 4.1 |
| Cetiol ® SN | Cetearyl | | 4.8 | | | | | | |
| | Isononanoate | | | | | | | | |
| Dow Corning ® | Cyclomethicone | 1.5 | — | 7.0 | 7.0 | 7.0 | 12.0 | 7.0 | 7.0 |
| 245 | | | | | | | | | |
| Eutanol ® G16 | Hexyldecanol | | 2.2 | | | | | | |
| Glycerin | | | 5.0 | 5.0 | 5.0 | 5.0 | 3.0 | 5.0 | 5.0 |
| Magnesium | | 0.8 | 1.0 | 1.0 | 1.0 | 1.0 | 1.0 | 1.0 | 1.0 |
| sulfate.7H₂O | | | | | | | | | |
| Aerosil ® R 972 | Silica Dimethyl | | | | | | 0.5 | 0.5 | 1.5 |
| | Silylate | | | | | | | | |
| Hydagen ® CAT | Triethyl Citrate | | | | | 1.0 | | | |
| Irgasan ® DP | Triclosan | | | | | 0.5 | | | |
| 300 | | | | | | | | | |
| Hydagen ® | Chitosan | | | | | 0.05 | | | |
| DCMF | | | | | | | | | |
| Rezal ® 36 GC | Aluminium Zirconium | | 40.0 | 40.0 | | | | | |
| | Tetrachlorohydrex | | | | | | | | |
| | Glycine | | | | | | | | |
| Locron ® L | Aluminium | 40.0 | | | 20.0 | | | 20.0 | 20.0 |
| | Chlorohydrate | | | | | | | | |
| Locron ® P | Aluminium | | | | | | 20.0 | | |
| | Chlorohydrate | | | | | | | | |
| Water | | to | to | to | to | to | to | to | to |
| | | 100 | 100 | 100 | 100 | 100 | 100 | 100 | 100 |
| Evaluation | | | | | | | | | |
| Microfoam | | − | + | + | + | + | + | + | + |
| Stability | | − | + | + | + | + | + | + | + |
| Absorption | | − | + | + | + | + | + | + | + |
| Tackiness | | 0 | + | + | + | + | + | + | + |
| Dryness | | 0 | 0 | 0 | 0 | 0 | + | + | + |
| Viscosity in | | 1750 | 1500 | 2000 | 2000 | 1500 | 2750 | 2500 | 3000 |
| mPa · s at 20° C. | | | | | | | | | |

Evaluation:
− (poor);
0 (average);
+ (good)
(Brookfield RVT, spindle TA, 20 r.p.m., with Helipath)

### APPENDIX

1) Aerosil® R 972
   INCI: Silica Dimethyl Silylate
   Manufacturer: Degussa AG
2) Arlacel® P 135
   INCI: PEG-30 Dipolyhydroxystearate
   Manufacturer: Uniqema (ICI Surfactants)
3) Cetiol® CC
   INCI: Dicaprylyl Carbonate
   Manufacturer: Cognis Deutschland GmbH
4) Cetiol® OE
   INCI: Dicaprylyl Ether
   Manufacturer: Cognis Deutschland GmbH
5) Cetiol® SN
   INCI: Cetearyl Isononaoate
   Manufacturer: Cognis Deutschland GmbH
6) Dehymuls® PGPH
   INCI: Polyglyceryl-2 Dipolyhydroxystearate
   Manufacturer: Cognis Deutschland GmbH
7) Dow Corning® 245
   INCI: Cyclomethicone
   Manufacturer: Dow Corning
8) Eumulgin® S2
   INCI: Steareth-2
   Manufacturer: Cognis Deutschland GmbH
9) Eumulgin® S21
   INCI: Steareth-21
   Manufacturer: Cognis Deutschland GmbH
10) Eutanol® G 16
   INCI: Hexyldecanol
   Manufacturer: Cognis Deutschland GmbH
11) Hydagen® DCMF
   INCI: Chitosan
   Manufacturer: Cognis Deutschland GmbH
12) Hydagen® CAT
   INCI: Triethyl Cltrate
   Manufacturer: Henkel KGaA
13) Irgasan® DP 300
   INCI: Triclosan
   Manufacturer: Ciba Specialty Chemicals Inc.
14) Locron® L
   INCI: Aluminium Chlorohydrate
   Manufacturer: Clariant GmbH
15) Locron® P
   INCI: Aluminium Chlorohydrate
16) Rezal® 36 GC
   INCI: Aluminium Zirconium Tetrachlorohydrex Glycine
   Manufacturer: Reheis Inc.

1-10. (canceled)

11. A composition comprising:
(a) an antiperspirant component;
(b) a polyolpoly-12-hydroxystearate;
(c) an oil component; and
(d) water
and wherein the composition is a water-in-oil emulsion having a viscosity of from about 500 to 10,000 mPas at 20° C.

12. The composition of claim 11 wherein the composition has a viscosity of from about 500 to 5,000 mPas at 20° C.

13. The composition of claim 11 wherein the antiperspirant component is an aluminium salt.

14. The composition of claim 11 wherein the antiperspirant component is present in the composition in an amount of from about 1 to 30% by weight, based on the weight of the composition.

15. The composition of claim 11 wherein the polyolpoly-12-hydroxystearate is present in the composition in an amount of from about 1 to 10% by weight, based on the weight of the composition.

16. The composition of claim 11 wherein the polyolpoly-12-hydroxystearate is present in the composition in an amount of from about 2 to 8% by weight, based on the weight of the composition.

17. The composition of claim 11 wherein the oil component is present in the composition in an amount of from about 5 to 30% by weight, based on the weight of the composition.

18. The composition of claim 11 wherein the oil component is present in the composition in an amount of from about 5 to 25% by weight, based on the weight of the composition.

19. The composition of claim 11 wherein the water is present in the composition in an amount of from about 40 to 90% by weight, based on the weight of the composition.

20. The composition of claim 11 wherein the oil component is selected from the group consisting of a dialkylether, a dialkylcarbonate, and mixtures thereof.

21. A process for inhibiting skin perspiration comprising contacting the skin with a composition containing:
(a) an antiperspirant component;
(b) a polyolpoly-12-hydroxystearate;
(c) an oil component; and
(d) water
and wherein the composition is a water-in-oil emulsion having a viscosity of from about 500 to 10,000 mPas at 20° C.

22. The process of claim 21 wherein the composition has a viscosity of from about 500 to 5,000 mPas at 20° C.

23. The process of claim 21 wherein the antiperspirant component is an aluminium salt.

24. The process of claim 21 wherein the antiperspirant component is present in the composition in an amount of from about 1 to 30% by weight, based on the weight of the composition.

25. The process of claim 21 wherein the polyolpoly-12-hydroxystearate is present in the composition in an amount of from about 1 to 10% by weight, based on the weight of the composition.

26. The process of claim 21 wherein the polyolpoly-12-hydroxystearate is present in the composition in an amount of from about 2 to 8% by weight, based on the weight of the composition.

27. The process of claim 21 wherein the oil component is present in the composition in an amount of from about 5 to 30% by weight, based on the weight of the composition.

28. The process of claim 21 wherein the oil component is present in the composition in an amount of from about 5 to 25% by weight, based on the weight of the composition.

29. The process of claim 21 wherein the water is present in the composition in an amount of from about 40 to 90% by weight, based on the weight of the composition.

30. The process of claim 21 wherein the oil component is selected from the group consisting of a dialkylether, a dialkylcarbonate, and mixtures thereof.
